(19) United States
(12) Patent Application Publication
(10) Pub. No.: US 2020/0005666 A1
(43) Pub. Date: Jan. 2, 2020

(54) REMOTE MENTORING STATION

(71) Applicant: THINK SURGICAL, INC., Fremont, CA (US)

(72) Inventors: Feimo SHEN, Fremont, CA (US); Peter NAKAMOTO, Fremont, CA (US); In K. MUN, Fremont, CA (US)

(21) Appl. No.: 16/348,478

(22) PCT Filed: Nov. 7, 2017
(86) PCT No.: PCT/US2017/060313

§ 371 Date: May 8, 2019

Related U.S. Application Data
(60) Provisional application No. 62/420,028, filed on Nov. 10, 2016.

(51) Int. Cl. G09B 19/00; G16H 40/67; H04L 29/08

(52) U.S. Cl. CPC G09B 19/00; H04L 67/125; G16H 40/67

(57) ABSTRACT

A system and method are provided that permits a mentor to remotely observe, train, or mentor an end-user of a medical device. The remote mentor is able to observe and take control, if necessary, of a medical device during a surgical procedure from a remote location using a network connection. New end-users, or end-users requiring assistance, may obtain feedback directly from the mentor in real-time. The remote mentor may remove power from the medical device at any time to provide assistance or recommendations to the end-user. The remote mentor can then re-activate the power, or allow the device to re-power, after assessing the situation.

### CROSS-REFERENCE TO RELATED APPLICATIONS

This application claims priority of U.S. Provisional Patent Application Ser. No. 62/420,028 filed Nov. 10, 2016 entitled “REMOTE MENTORING STATION”, the entire contents of which is incorporated herein by reference in its entirety.

### TECHNICAL FIELD

The present invention generally relates to the field of computer-assisted surgery, and more specifically to a system and method to remotely mentor an end-user of a medical device during computer-assisted surgery.

### BACKGROUND

Training a surgeon and their medical team with a new and complex medical device is both time consuming and requires on-site trainers in the operating room (OR) for several procedures. Depending on the complexity of the device, an experienced mentor (i.e., a user, surgeon, or trainer having extensive experience using the device) is recommended to train the new user. This is especially the case in robotic surgery, where the new user requires several days of training and supervised patient cases before becoming proficient with the device. When a new device is first released on the market or when there are only a few devices in the field, the number of experienced mentors is low. Thus, the mentors usually have to travel long distances and expense their time to train new users in the OR and repeat the inefficient travel to the next treating OR. This travel and training time also hinders the manufacturer's ability to globally release the product in an efficient time frame.

In other situations, a first experienced end-user may simply want to obtain additional advice from a second experienced end-user. The first end-user may want to learn additional skills with the device, obtain a second opinion for a particular patient case, or discuss possible innovations using the device. For example, an end-user may be capable of proficiently executing a standard total hip arthroplasty (THA) with no complications. However, if the end-user has a patient with severe hip dysplasia, the end-user may want to consult with a mentor having experience with hip dysplasia cases. The end-user may even want to have the mentor available during the procedure. In such a situation, the mentor has to either provide advice to the end-user prior to the procedure, or travel to the location of the patient and physically be present in the OR.

Finally, with respect to robotic surgery, the end-user must have the ability to pause or stop the robotic arm at any point during the procedure. Even though robotic devices are designed with several fail-safe mechanisms, the end-user must always supervise and have complete control of the robotic device. Control of the robotic device is typically accomplished using an input device (e.g., pendant, controller, joystick) that allows the user to immediately remove power to the robotic arm, among other functions. This is an important safety requirement for any robotic system. If the end-user is being trained by a mentor, the mentor should also have the ability to control or remove power from the device, which requires the physical presence of the mentor in the OR.

Thus there is a need in the art for a system and method that permits a mentor to remotely observe, train, or mentor an end-user of a medical device. There is a further need for the remote mentor to have the ability to control or quickly cut power to the device.

### SUMMARY

A remote mentoring system is provided that includes a medical device located in an operating room (OR), and a remote mentoring station located outside the OR having a remote display of the medical device and a remote input device. A network connection provides the remote mentoring station with observation of the medical device and communication to the OR thereby enabling a mentor to provide an end-user of the medical device instruction and to provide the remote mentor using the remote mentoring station control to the medical device via the remote mentoring station.

A method of using a remote mentoring system for conducting medical procedures is provided. The method includes connecting the remote mentor station outside an operating room to the medical device in the operating room (OR), and using the remote mentor station to communicate with and control the medical device.

### BRIEF DESCRIPTION OF THE DRAWINGS

The present invention is further detailed with respect to the following drawings that are intended to show certain aspects of the present of invention, but should not be construed as limit on the practice of the invention, wherein:

FIG. 1 depicts a diagram of remote mentoring station and operating room where a remote mentor can control a medical device in accordance with embodiments of the invention;

FIG. 2 depicts a diagram of a remote mentoring station and operating room where a remote mentor can control the power of a medical device in accordance with embodiments of the invention; and

FIG. 3 depicts a diagram of a remote mentoring station and operating room where a remote mentor can control the power of a robotic arm in accordance with embodiments of the invention.

### DETAILED DESCRIPTION

The present invention has utility as a system and method that permits a mentor to remotely observe, train, or mentor an end-user of a medical device. Embodiments of the present invention allow the remote mentor to observe and take control, if necessary, of a medical device during the surgical procedure from a remote location using a network connection. Therefore, new end-users, or end-users requiring assistance, may obtain feedback directly from the mentor in real-time. Specifically, the remote mentor may remove power from the medical device at any time to provide assistance or recommendations to the end-user. The remote mentor can then re-activate the power, or allow the device to re-power, after assessing the situation. The following description of the various embodiments of the invention is not intended to limit the invention to these specific embodiments, but rather to enable any person skilled in the art to make and use the invention through exemplary aspects thereof.

As used herein, a medical device is to encompass any device used in a surgical operation or used in the diagnostics of a patient or subject. In particular embodiments, the medical devices are computer-assisted surgical systems including, but not limited to a hand-held surgical system having 1, 2, . . . N degrees of freedom as to movement, an autonomous serial-chain manipulator system, a haptic serial-chain manipulator system, a parallel robotic system, a master-slave robotic system, or navigated surgical instruments, such as those described in U.S. Pat. Nos. 5,086,401, 7,206,626, 8,876,830, 8,961,563, U.S. Pat. App. No. 2013/0060278, and PCT Intl. App. No. US2015/051713. Examples of robotic systems particularly useful with embodiments of the invention include The TSolution One® Surgical System (THINK Surgical, Fremont, Calif.), the RIO® Robotic Arm Interactive Orthopedic System (Stryker-Mako, Ft. Lauderdale, Fla.), the ROSA™ Robotic Device (Zimmer-Medtech, Montpellier, France), and other robotic surgical systems having an electromechanical arm.

While depicted in the following drawings as a single OR, it is appreciated that in some inventive embodiments, a monitor station is coupled to a plurality of ORs, thereby allowing a remote mentor to be available as a resource during multiple medical procedures, simultaneously or sequentially, using the medical device. This is exploited as a business method, in which the remote mentor access is provided for financial remuneration as a consultant during the planning of a procedure involving the medical device, during the procedure, or post-operatively.

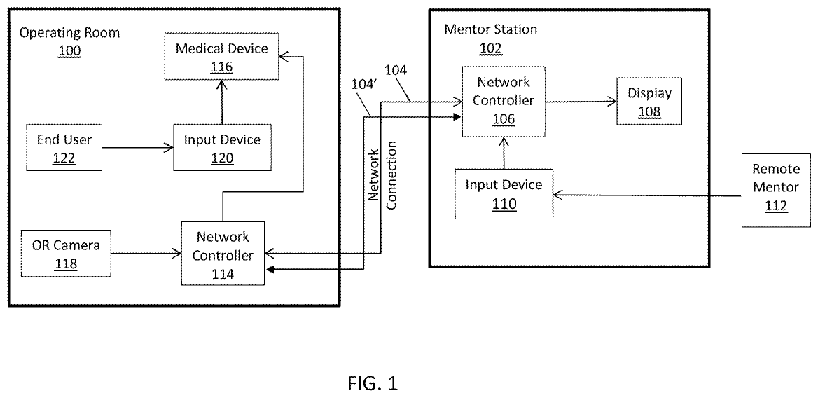

Referring now to the figures, FIG. 1 is a diagram of a remote mentor station 102 and an operating room (OR) 100 connected by a network connection 104. The remote mentor station 102 is located outside of the operating room 100 and includes a remote network controller 106, a remote display 108, and a remote input device 110. A remote mentor 112 uses the remote mentor station 102 to communicate with and control the medical device 116 in the operating room 100.

In a particular embodiment, the operating room 100 includes an OR network controller 114, a medical device 116, an OR camera 118, an OR input device 120, and the end-user 122. The network connection 104 transmits data between the mentor station 102 and the operating room 100. The medical device 116 selectively engaging a surgical field of a patient. In a particular embodiment, the network connection 104 is any type of known network including a fixed wire line network, cable and fiber optics, over the air broadcasts, satellite, local area network (LAN), wide area network (WAN), global network (e.g., Internet), intranet, etc. that have high transmission data rates and low latency. Additional forms of network connections are also viable including wireless data transfer with Wi-Fi and Bluetooth, or wired data transfer such as an Ethernet line. In some inventive embodiments, a redundant network connection 104′ is provided to mitigate a network outage during a surgical procedure in the OR 100. The redundant network connection 104′ ideally being on a separate carrier to harden the connection against an outage of a given network. However, it will become apparent that the data rates and latency between the OR 100 and the mentor station 102 are important in certain embodiments.

The mentor station 102 may be located in the same hospital as the OR 100 or located as far away as another country. The OR network controller 114 and remote network controller 106 process and control the incoming and outgoing data. In a particular embodiment, the OR camera 118 (e.g., CCD, CMOS, or other video camera imaging source) provides a live-feed to the remote display 108 (e.g., video monitor, computer monitor, tablet display, smartphone, and the like). The remote mentor 112 may then watch the procedure in real-time on the remote display 108. In a specific embodiment, the remote mentor 112 may provide input to the medical device 116 using an input device 110. In a specific embodiment, the input device 110 is a single button that allows the remote mentor 112 to remove power from the medical device 116 as further described below. The use of a single button or other touch sensitive control as the input device 110 allows the mentor to only have the ability to stop the procedure and provide feedback to the end-user 122 without taking complete control over the device. This is more akin to a traditional mentoring approach (i.e., stop and teach), rather than having the mentor perform the actual surgery (i.e., take full control of the device). However, it should be appreciated that the input device 110 may have additional inputs to control the medical device 116. For example, the input device 110 may include a joystick, a mouse, a pendant, a keyboard, a foot pedal, or a specifically designed input device having several buttons for controlling several different functions of the device (i.e., a space mouse). In a specific embodiment, the input device 110 may be integrated with the display device 108 as a touchscreen with the ability to enter commands via the display screen. In other inventive embodiments, the mentor has a stereoscopic display that provides a field of view depth that appears 3-dimensional. In still other embodiments, the mentor manual input device provides haptic feedback as the medical device 116 interacts with patient tissue.

The end-user 122 also has control over the medical device 116 via the OR input device 120. In a particular embodiment, the OR input device 120 has additional functionality than that of the remote input device 110. Therefore, in this particular embodiment the end-user 122 has greater control over the medical device 116 and the procedure in general. In another embodiment, the OR input device 120 and the remote input device 110 are identical. In a specific embodiment, safety mechanisms are employed in the event the network connection is lost. In such an event, the end-user 122 would take full control over the medical device 116 for safety and disconnect any inputs from the remote mentor 112.

In a specific embodiment, the medical device 116 has direct network connection capabilities (Internet communication), wherein the network controller 114 is essentially embodied within the medical device 116.

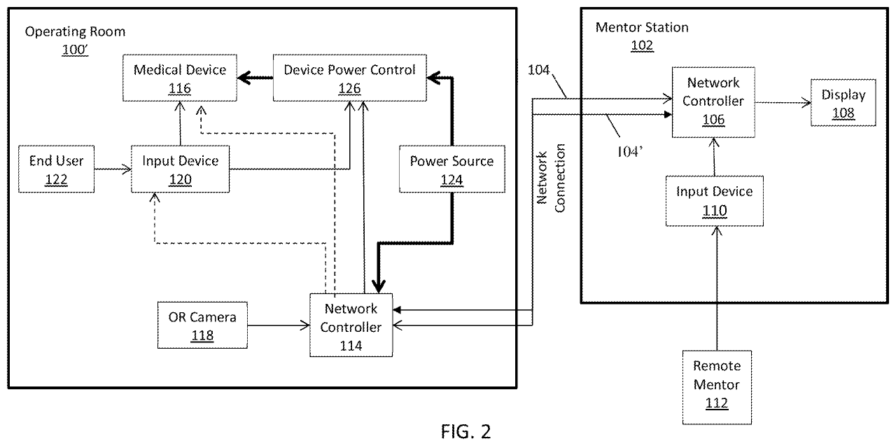

With reference to FIG. 2, a specific embodiment of a remote mentor station 102 and an operating room 100′ is shown. The operating room 100′ now includes a power source 124 and a device power control unit 126. The power source 124 may be a typical A/C outlet present in the OR, or another external power source. The device power control unit 126 may be a power relay, or a circuit switch, that controls the power to the medical device 116. In a specific embodiment, the remote mentor 112, via remote input device 110, can directly control the power to the device 116. For instance, if the remote mentor 112 notices the end-user using the medical device 116 in an ill-advised or dangerous fashion, the remote mentor 112 can activate a circuit switch, by pressing a button, a switch, or by voice activation (associated with the input device 110), causing the circuit switch to open to remove power from the device 116. The remote mentor 112 can then provide feedback to the end-user 122 via a phone call, a video call, an audible speaker present in the OR, or by mirroring data provided by the remote mentor on the display 108 to a display present in the OR 100′. Once the feedback is conveyed, the remote mentor 112 can disengage the circuit, closing the switch 126, to re-activate power to the medical device 116 or to permit the end-user to re-power the device 116. As shown in FIG. 2, the dark bold lines represent power lines, the non-bold lines represent data flow, and the dashed lines represent optional data flow.

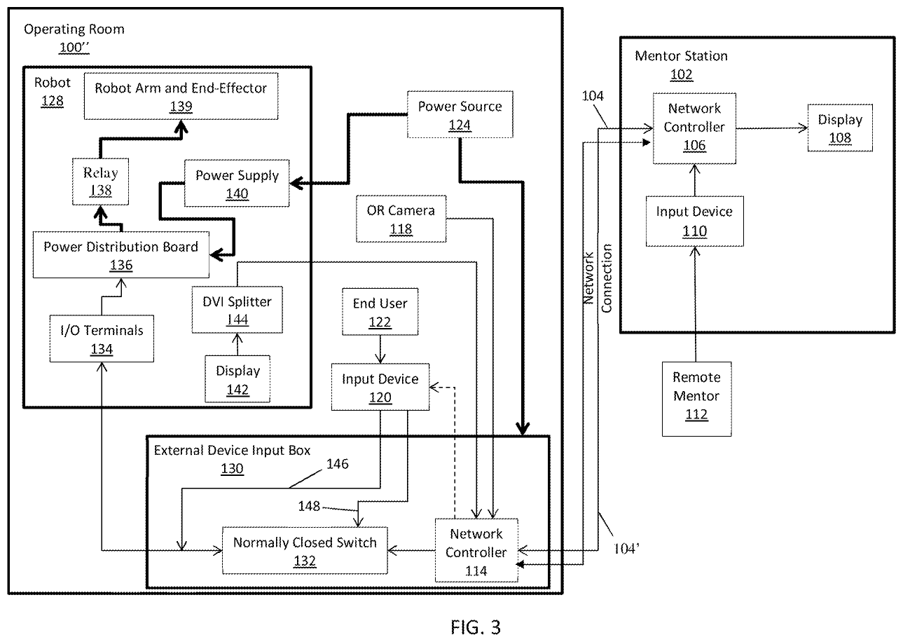

With reference to FIG. 3, a specific embodiment of an operating room 100″ and remote mentor station 102 is shown. The OR 100″ includes a robotic system 128 and an external device input box 130. The robotic system 128 includes input-output (I/O) terminals 134, a power distribution board 136, a power relay 138, a robot manipulator arm having an end-effector 139, a power supply 140, a robot display 142, and a digital visual interface DVI splitter 144. The external device input box 130 houses circuitry including a normally closed switch 132, and the OR network controller 114. The normally closed switch 132 provides a mechanism for both the end-user 122 via OR input device 120, and the remote mentor 112 via remote input device 110, to directly remove power from the robot arm and/or end-effector 139. In a particular embodiment, the OR input device 120 controls a first normally closed switch, and the remote input device 110 controls a second normally closed switch, where both the first switch and second switch are within the same circuit. The input devices 110, 120 have an emergency power-off button, which when activated opens the normally closed switch(s) 132. When the switch opens, the I/O terminal 134 processes the removed voltage and immediately removes power from the robot arm and/or end-effector 139. The emergency power-off line for the OR input device 120 is shown at 148. Other input signals generated from the OR input device 120 are routed through the line shown at 146. As before, the dark bold lines represent power lines, the non-bold lines represent data flow, and the dashed lines represent optional data flow. In a specific embodiment, activating the emergency power-off only removes power from a portion of the robotic system 128. For example, the emergency power-off may only remove power from the robotic arm and/or the end effector 139, while still maintaining power to a base of the robotic system 128, or to a display 142 connected to the system 128.

The OR display 142 is in communication, by a wired or wireless connection, to the medical device 116, such as the robot 128 of FIG. 3. The OR display 142 may guide the end-user 122 through a procedure, display images of the anatomy, provide navigation data, provide workflow instructions, provide robot operational data, allow the end-user 122 to interact with the medical device 116 and provide inputs to the medical device 116. In a specific embodiment, the images or video on the OR display 142 are routed to the mentor display 108. The remote mentor 112 can watch what the medical device is displaying on the OR display 142 and watch, in real-time, how the end-user interacts with the medical device 116 through the OR display 142. In a specific embodiment, the remote mentor 112 may have a split screen of the OR camera 118 and the OR display 142 in order to watch both video streams simultaneously.

### Other Embodiments

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the described embodiments in any way. Rather, the foregoing detailed description will provide those skilled in the art with a convenient roadmap for implementing the exemplary embodiment or exemplary embodiments. It should be understood that various changes may be made in the function and arrangement of elements without departing from the scope as set forth in the appended claims and the legal equivalents thereof.

1. A remote mentoring system comprising:
a medical device located in an operating room (OR);
a remote mentoring station located outside the OR having a remote display of said medical device and a remote input device; and
a network connection providing said remote mentoring station with observation of said medical device and communication to the OR providing an end-user of said medical device instruction and to provide a remote mentor using said remote mentoring station control to said medical device via said remote mentoring station.

2. The system of claim 1 wherein said remote display receives a live feed of a surgical field entered by said medical device.

3. The system of claim 1 further comprising a redundant network connection.

4. The system of claim 1 wherein said remote input device is a single power off apparatus to dis-engage said medical device.

5. The system of claim 1 wherein said remote input device is at least one of: a joystick, a mouse, a pendant, a keyboard, a foot pedal, a voice control, or a specifically designed input device having several buttons for controlling several different functions of said medical device.

6. The system of claim 1 wherein said remote input device and said remote display receive a live feed of a surgical field or procedure field involving said medical device, where said input device is integrated with said remote display device as a touchscreen with the ability to enter commands via said remote display for control of said medical device.

7. The system of claim 1 further comprising an OR input device that has additional functionality relative to said remote input device providing the end-user with greater control over said medical device as compared to the mentor.

8. The system of claim 1 further comprising a safety mechanism that is employed in the event the network connection is lost, where if the network connection is lost, the end-user takes full control over said medical device, and the safety mechanism disconnects any inputs from the remote mentor.

9. The system of claim 1 further comprising a device power control unit in the OR that acts as a power relay, or a circuit switch, that controls the power to said medical device.

10. The system of claim 1 wherein said medical device is a surgical robot.

11. The system of claim 1 wherein said remote display is stereoscopic.

12. The system of claim 1 wherein said remote input is a haptic control.

13. The system of claim 1 wherein said network connection has a high transmission data rate and a low latency.

14. The system of claim 1 wherein said remote mentoring station is coupled to a plurality of operating rooms allowing the remote mentor to be available as a resource during multiple medical procedures, simultaneously or sequentially, using the medical device.

15. A method of using the system of claim 1 comprising:
connecting the remote mentor station outside an operating room to the medical device in the operating room (OR); and
using the remote mentor station to communicate with and control the medical device.

16. The method of claim 15 wherein a mentor using the remote mentor station is located in a hospital housing the operating room or the mentor is located as far away as another country.

17. The method of claim 15 wherein a mentor watches a live-feed of a procedure on a remote display conducted with the medical device.

18. The method of claim 17 wherein the remote display comprises a video monitor, computer monitor, tablet display, or a smartphone.

19. The method of claim 15 further comprising removing power from the medical device using a single button thereby allowing the mentor to provide feedback to an end-user without taking complete control of the medical device.

20. The method of claim 15 further comprising the mentor controlling the medical device with an input device.
